(19) United States
(12) Patent Application Publication
(10) Pub. No.: US 2012/0305003 A1
(43) Pub. Date: Dec. 6, 2012

(54) RAPID PRODUCTION OF CUSTOMIZED MASKS

(76) Inventor: Phillip E. Mark, Orlando, FL (US)

(21) Appl. No.: 13/498,224

(22) PCT Filed: Oct. 21, 2009
(86) PCT No.: PCT/US09/58214

§ 371 Date: Aug. 23, 2012

(51) Int. Cl. A61M 16/06; G06F 17/50

(52) U.S. Cl. 128/206.24; 703/1

(57) ABSTRACT

Disclosed herein is a system designed for the rapid preparation of anatomically customized mask employing data from a patient. The data may take the form of a multidimensional image of a target area of a patient's face obtained by optical 3 dimensional imaging, or a dot or line scan form laser imaging, pattern laser photography or stereo photography. Also disclosed is a mask that is designed so as to be unobtrusive and comfortable for the user. The body of the mask is made of a thin layer, so it is lightweight and closely hugs the targeted region upon which it rests (e.g. the nasal region). Methods for producing anatomically customized masks are also described.

### BACKGROUND

Obstructive sleep apnea (OSA) and other sleep-related respiratory disorders are debilitating conditions that, if left untreated, can result in dramatic health consequences. Obstructive sleep apnea is characterized by instability of the upper airway occurring during sleep and leads to frequent episodes of breathing cessation (apnea) or decreased airflow (hypopnea). During these episodes, the patient has a brief arousal from sleep that allows restoration of airway patency and resumption of breathing. The segmentation of sleep derived from these episodes of “nocturnal asphyxia”, which can occur as much as 400-500 times per night, leads to excessive daytime somnolence and narcolepsy. Hypersomnolence can become disabling and dangerous; studies show that patients with OSA have two to seven times more motor vehicle accidents than people without OSA. In addition, these episodes can also cause intellectual impairment, memory loss, personality disturbances, impotence, arrhythmias, hypertension, heart attacks, stroke, and premature death. Surgical and non-surgical therapies have been developed for alleviating the symptoms of sleep apnea, or even curing sleep apnea. While surgery may make sense in extreme cases, the surgery can be expensive, and painful, and the surgical outcomes are not always positive. The most common non-surgical therapy for sleep apnea is continuous positive airway pressure (CPAP). One problem that the inventor has now realized with respect to CPAP is that the equipment used is uncomfortable, which affects the ability to sleep. In addition, CPAP masks and equipment are unsightly, which has a negative psychological result concerning one's own well-being.

### BRIEF DESCRIPTION OF THE DRAWINGS

FIGS. 1a-d show a diagram showing a system embodiment of the invention;

FIG. 2 shows an image of a point cloud useful with an embodiment of the present invention;

FIG. 3 shows a stereo_lithograph image useful with an embodiment of the invention;

FIG. 4 shows a stereo_lithograph image useful with an embodiment of the invention;

FIG. 5 shows an image depicting regions of the face that are scanned according to an embodiment of the invention

FIGS. 6 a-e show a pictorial flow diagram of a method of the invention; and

FIG. 7 shows an image depicting imaging of a face according to an embodiment of the invention.

FIG. 8 shows a top perspective view 8a, a back perspective view 8b, a top and planar view 8c of another embodiment of the invention.

FIG. 9 shows a bottom elevational view of the embodiment shown in FIG. 8.

FIG. 10 a-c shows a schematic representing a mold making embodiment of the invention.

FIGS. 11 and 12 show a schematic representing a mold, making embodiment of the invention.

FIGS. 13-15 show a schematic representing a mold, making embodiment of the invention.

FIG. 16 shows a top perspective view 16a and a back perspective embodiment 16b of an embodiment of the invention.

FIG. 17 shows a projector with dual cameras for obtaining images of a patients face.

FIG. 18 shows a further embodiment showing producing of a mask with a customized seal attached to a generic mask body.

FIG. 19 shows a mask body comprising a first layer and a second layer of material having increased insulating potential.

### DETAILED DESCRIPTION

The inventor has realized that while some efforts have been made to improve the comfort of conventional CPAP masks by providing a number of different sizes, comfort levels still fall short. The invention is based on the inventor's discovery that the comfort level of CPAP masks has a dramatic effect on the compliance and effectiveness of CPAP for treating OSA. In one embodiment, the invention pertains to the rapid production of masks used in conjunction with CPAP that are customized to a particular region of a patient's face. The methods of making customized masks and the configuration of mask embodiments disclosed herein dramatically improves the comfort level of masks.

In one embodiment, the invention is directed to a system designed for the rapid preparation of anatomically customized mask employing data from a patient. The data may take the form of a multidimensional image of a target area of a patient's face obtained by optical 3 dimensional imaging, or a dot or line scan form laser imaging, pattern laser photography or stereo photography.

The data is typically acquired at a first site, while engineering and/or manufacturing services and equipment are located at a second site, remote with respect to the first site. Transmittal of a patient's data over telecommunication or computer networks can significantly reduce the time required for production and manufacture of masks.

For processes using a mold to produce the mask, such as through vacuforming techniques, the mold is formed to provide certain adjustments to the configuration of the mask to improve comfort and fit of the mask. For example, the mold is made to have a protruding surface (or raised geometry) such that a plenum between the inner surface of the mask and a portion of the target region is formed. This could be accomplished, for example, by modifying the scanned image or multidimensional image to generate a protruding surface that extends from the natural contour of a portion or the entire target region. In a specific embodiment, the protruding surface may extend between 0.1-1.0 inches off of the natural contoured surface of the target region. In a more specific embodiment, the protruding surface extends between 0.15-0.4 inches off the natural contoured surface of the target region. In an even more specific embodiment, the protruding surface extends about 0.25 inches off the natural contoured surface of the target region. A layer of plastic or the like, is vacuformed over the mold, removed from the mold and cut. A silicon or thermoplastic ring is made to fit the outer edge of the mask. This ring will be incontact directly with the patient's face and will allow a wide range of facial movement, while still retaining the seal.

In one embodiment, an image is produced from the dataset, which in turn is used to produce a mold of the target region. The mold is then used to produce a mask specifically tailored and customized to cover the target region of the patient, from which data was obtained. In one embodiment, the mold is formed using a computer numerical control (CNC) machine that forms the mold based on the dataset.

In certain embodiments, the mask is imprinted or engraved with the patient's name and a serial no. is assigned to the mask. The serial no. is catalogued and stored in a database such that additional masks may be reordered for the patient. Also, the mask may engrave or imprinted with marketing information such as trademarks or logos of suppliers. In this sense, the mask also serves as potential marketing and promotional function.

One advantageous feature of the mask is that it is designed so as to be unobtrusive for the user. The body of the mask is made of a thin layer, so it is lightweight and closely hugs the targeted region upon which it rests (e.g. the nasal region). In a specific embodiment, the thin layer material is 3/32 inches to ⅛ inches in thickness. In another specific embodiment, the mask is lightweight, between 15 to 45 grams. Also, the mask is configured to key on the nasal bridge and follows the contours of the nasal bridge so as to minimize cantilevering of the mask at the nasal bridge. Thus, if pressure is applied to either the left or right sides of the mask, the mask does not lift off the nasal bridge. In addition to having the portion of the mask closely simulate the geometry of the nasal ridge, the mask also closely simulates the patient's upper lip just under the patient's nares. In addition, the lower portion of the sides of the mask (about the lower quarter portion) closely simulate the geometry of the patient's face. Thus, the nasal ridge portion of the mask, the lower side portions, and the bottom portion under the nose farm a four-point contact that stabilizes the mask on the patient's face. As pointed out above, the rest of the nose does not contact the mask, thereby allowing the nose to flare without touching the inner walls of the mask. Also, the plenum between the inner wall of the mask and the patient's nasal surface allows the nasal surface to be cooled by air in the mask, which avoids uncomfortable sweating of the nasal surface.

### EXAMPLES

### Example 1

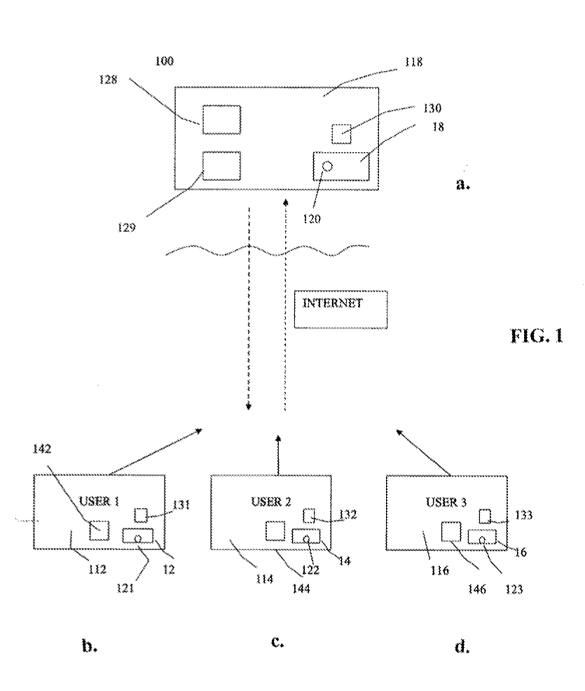

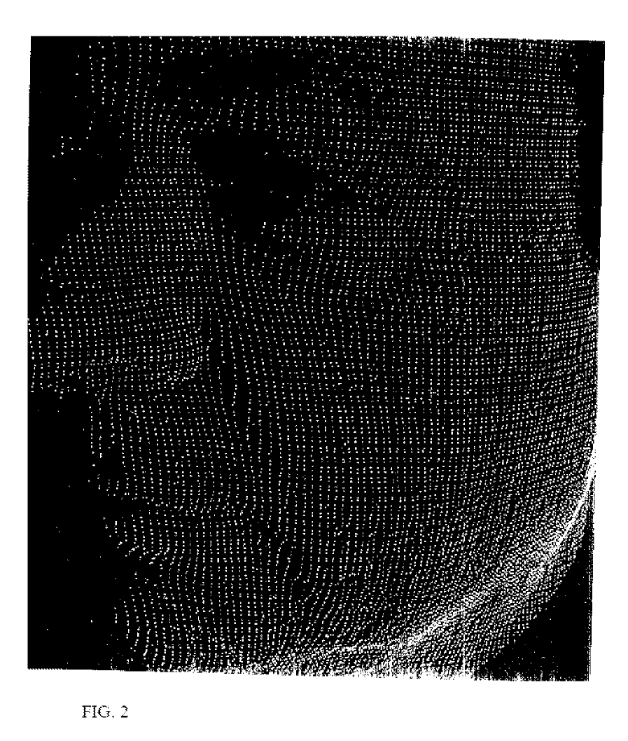

FIG. 1 and the following discussion provide a brief, general description of a suitable computing environment in which embodiments of the invention can be implemented. Although not required, embodiments of the invention will be described in the general context of computer-executable instructions, such as program application modules, objects, or macros being executed by a computer. Those skilled in the relevant art will appreciate that the invention can be practiced with other computer system configurations, including hand-held devices, multiprocessor systems, microprocessor-based or programmable consumer electronics, personal computers (“PCs”), network PCs, mini computers, mainframe computers, and the like. The invention can be practiced in distributed computing environments where tasks or modules are performed by remote processing devices, which are linked through a communications network. In a distributed computing environment, program modules may be located in both local and remote memory storage devices.

Referring to FIG. 1, a rapid customized mask design and manufacturing system 100 includes a client scanning/computing system 112, 114, and 116 and a host computing/mask producing system 118. The client scanning/computing system 12, 14, and 16 may be located at a diagnostic site, such as a hospital, clinic, laboratory or doctor's office. The host computing system 18 may be located at a site remote from the diagnostic site, such as at a site of a manufacturer. Client scanning/computer systems 112, 114, 116 each having a user computer unit 12, 14, 16 that interface with host computer unit 18 via a network connection 109. Each computer unit comprises at least one processing module 120, 121, 122, and 123, respectively, for processing information. Furthermore, each computer unit is communicatingly connected to a display 130 (optional), 131, 132, and 133. Communicatingly connected with each computer unit 12, 14, and 16 is a scanner 142, 144, and 146 for acquiring scanned images of patients target region of the face. The scanned images are processed by computer units 12, 14, and 16 and sent to host computer unit 18. The host computer unit processes image and directs the production of a mold via a CNC machine 128 based on the image data received from client 112, 114, and 116. Upon production of a mold, a mask is made via a vacuforming machine 129 that utilizes the mold made by the CNC 128 machine.

Alternatively, the client/scanning computers are not needed if the scanning and cutting take place in one location. In this alternative embodiment, a scanned image of a target region is obtained and then a CNC is directed to produce a mold as controlled by a single computer.

### Example 2

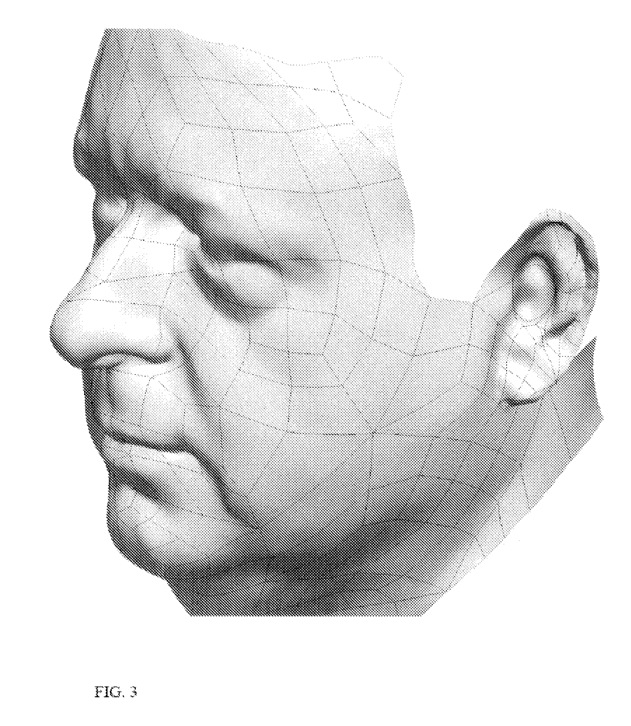

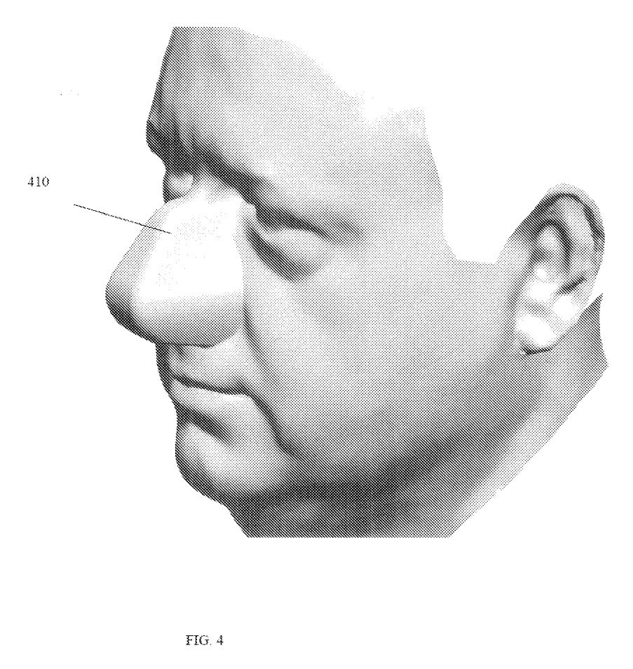

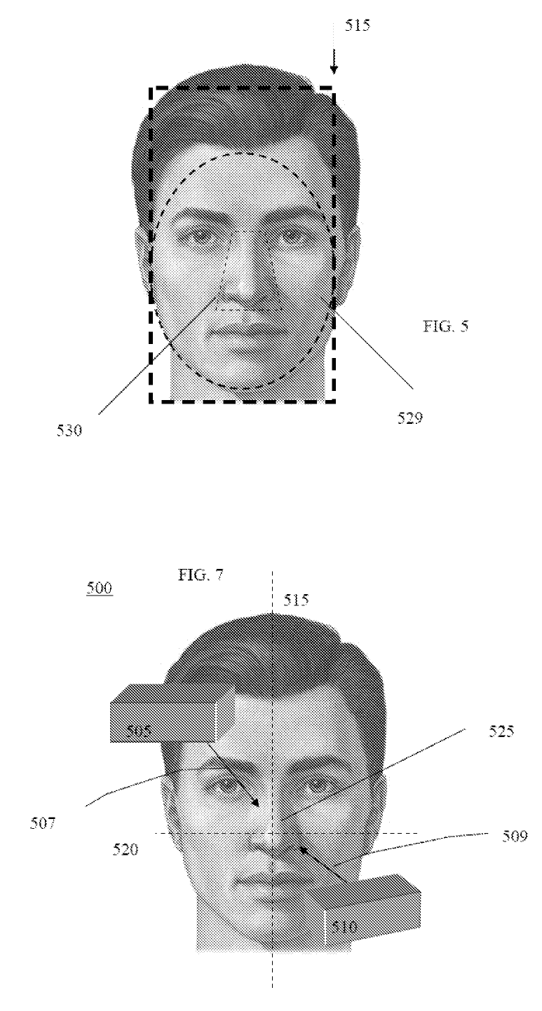

In a specific embodiment, the contour dataset from the scan(s) is used to form a sterolithography (STL) model image which is used by the CNC to form the mold according to known techniques. In a more specific embodiment, the contour dataset from the scan(s) relates to a point-cloud as shown in FIG. 2, which is used to form a stereolithography model image (FIGS. 3 & 4), which is used by the CNC to form the mold according to known techniques. Shown in FIG. 4 is the protrusion modification 410 to the image. This will direct production of the plenum during the mask forming process described below.

### Example 3

According to another embodiment, the invention pertains to a method of rapidly preparing a mask customized to a portion of a patient's face. The method entails the scanning of a patient's face contemporaneously from a first scanning angle and a second scanning angle. Referring to FIGS. 5 and 7, a specific method embodiment 500 is shown, wherein the first scanning head 505 scans at a first scanning angle 507 and second scanning head 510 scanning at a second scanning angle 509, wherein the scanning angles are oblique to a coronal plane 515 of a patient's head. As to the embodiment shown in FIG. 5, the first scanning angle 507 is at an angle above a horizontal plane 520 passing through the nose 525 of the patient, and the second angle 509 is at an angle below the horizontal plane 520. This specific embodiment 500 enables data to be obtained pertaining to the contours of a patient's target scan region 529 from both an upper and lower angle in one scanning step. The scans are integrated to provide a dataset corresponding to the contours of a target facial region, such as a nasal region 530. In turn, this allows for accurate contouring of regions under the nose that are not achieved by a single uniaxial scan direction. Though not shown, the scanning method preferably scans the target region while the patient is in a supine position. This is done to better reflect the affect of gravity on the features of the face, which in turn will make the mask more comfortable and form fitting to a sleeping patient. In an alternative embodiment, two separate scans are produced: one from an angle above a horizontal plane of a patient's face and another below the horizontal plane. These two scans are integrated to form a dataset corresponding to the contours of a patient's nasal region.

### Example 4

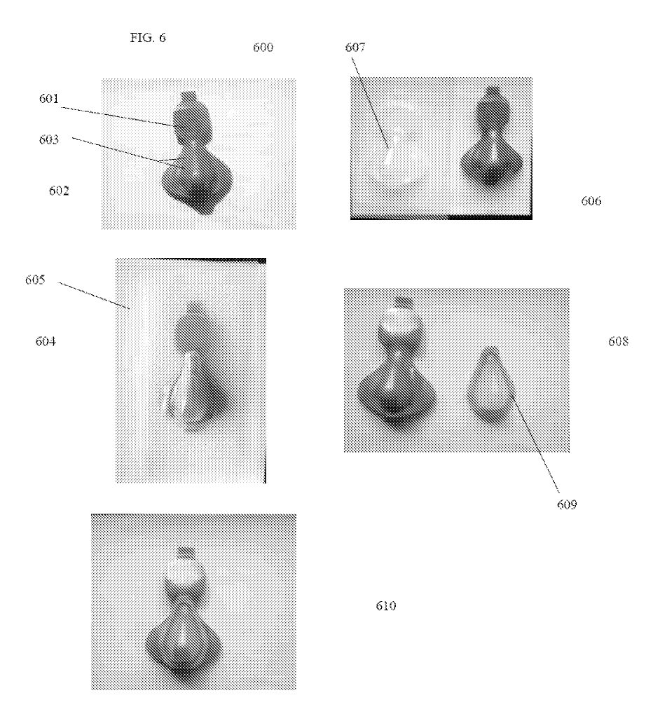

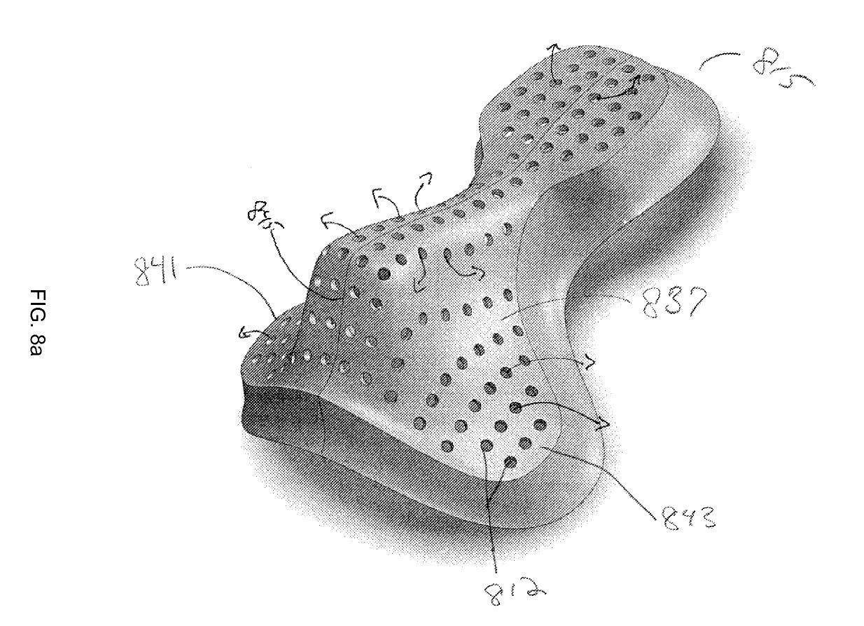

Turning to FIG. 6, a method 600 for manufacturing a mask is presented pictorially. In a first step (shown in 602), a mold 601 of target region of a patient's face is made. In this example, the mold 601 is made based on the stereolithography model shown in FIGS. 3 & 4. Thus, the mold 601 has a raised geometry 603 for creating the plenum as discussed above. Upon forming the mold 601, a thin layer of a plastic 605 (or other suitable material such as silicon) is placed over the mold and formed over the mold (shown in 604), such as by heating and vacuforming. The plastic material 605 is removed from the mold (shown in 606). The plastic layer 605 is cut to form a mask 607. A sealing material 609, of moderate durometer material such as silicon or rubber, is applied to the periphery of the cut mask (shown in 608). The sealing material 608 at the periphery of the mask 607 will contact with the patient's face (as shown in 610). The areas of the mask that do not come into contact with skin will include apertures and slots to accommodate connection of hoses for delivery of air and straps to secure the mask to the face As will be described further below, techniques other than vacuforming using a mold, such as stereolithography, may be used to form the mask.

As will be appreciated by one of skill in the art, embodiments of the present invention may be embodied as a device or system comprising a processing module, and/or computer program product comprising at least one program code module. Accordingly, the present invention may take the form of an entirely hardware embodiment or an embodiment combining software and hardware aspects. Furthermore, the present invention may include a computer program product on a computer-usable storage medium having computer-usable program code means embodied in the medium. Any suitable computer readable medium may be utilized including hard disks, CD-ROMs, DVDs, optical storage devices, or magnetic storage devices.

The term “processing module” may include a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The processing module may have operationally coupled thereto, or integrated therewith, a memory device. The memory device may be a single memory device or a plurality of memory devices. Such a memory device may be a read-only memory, random access memory, volatile memory, non-volatile memory, static memory, dynamic memory, flash memory, and/or any device that stores digital information. A computer, as used herein, is a device that comprises at least one processing module, and optionally at least one memory device.

The computer-usable or computer-readable medium may be or include, for example, but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM), a CD ROM, a DVD (digital video disk), or other electronic storage medium. Note that the computer-usable or computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory.

Computer program code for carrying out operations of certain embodiments of the present invention may be written in an object oriented and/or conventional procedural programming languages including, but not limited to, Java, Smalltalk, Perl, Python, Ruby, Lisp, PHP, “C”, FORTRAN, or C++. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or a wireless network, or the connection may be made to an external computer (for example, through the Internet using an Internet Service Provider).

Certain embodiments of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer-readable program code modules. These program code modules may be provided to a processing module of a general purpose computer, special purpose computer, embedded processor or other programmable data processing apparatus to produce a machine, such that the program code modules, which execute via the processing module of the computer or other programmable data processing apparatus, create means for implementing the functions specified in the flowchart and/or block diagram block or blocks.

These computer program code modules may also be stored in a computer-readable memory that can direct a computer or other programmable data processing apparatus to function in a particular manner, such that the program code modules stored in the computer-readable memory produce an article of manufacture.

The computer program code modules may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart and/or block diagram block or blocks.

Encryption, password protection and digital certificate authentication is desirable in any such data transmission. Transmission of approval from the physician to the manufacturer can be stored with the file containing the agreed-upon design, forming a record of the same.

As discussed above, the customized masks may be constructed utilizing a mold and vacuforming process. However, masks may be constructed using new modified rapid prototyping techniques. Conventional three-dimensional printing (3DP) techniques, such as stereolithography, involve selectively bonding together powder in successively deposited layers to form generalized solid shapes. Rapid prototyping processes are detailed in U.S. Pat. Nos. 5,204,055, 5,387,380, 5,807,437, 5,340,656, 5,490,882, 5,814,161, 5,490,962, 5,518,680, and 5,869,170, all hereby incorporated by reference.

Since three-dimensional printing involves printing in layers, it requires instructions in which a multi-dimensional digital model is mathematically translated into a series of slices of narrow thickness, each slice having a set of data or printing instructions representing the part geometry at that particular plane. In three-dimensional printing, each slice corresponds to a layer of powder/liquid in the bed during construction of the model. The entire set of data or instructions is referred to as the machine instructions.

The present invention's use of an electronic design and manufacturing masks also permits additional advantages such as compilation of databases or profiles for individual physicians and/or hospitals or for individual patients, inventory control, record-keeping and billing, product design updates and client feedback, branding for selected distributions, and follow-up notices to users. Such information can be maintained on a secure Web site, available to appropriate categories of users such as through the use of passwords or similar access restrictions.

In some instances, the present invention may be used in a way which does not involve constructing masks to order, but rather involves selecting the best fit from a stock of already-manufactured components or designs. While selection from stock does not provide all of the advantages of manufacturing completely customized parts to order, it nevertheless would provide some degree of customization that might be adequate for certain purposes. It also would be even faster than fully customized manufacture. In this sort of application, scan data pertaining to a specific patient could still be employed, and could assist in deciding which stock item should be used. The selected stock items are shipped to the physician. This could executed by a central website would have further usefulness in that it could be used for maintaining records of inventory, records of rates of use, patterns of deterioration for subsequent product improvement design adjustments, and could indicate the need for replenishing items which are out of stock or nearly out of stock. Of course, similarly, for custom manufacturing, the website could still help to maintain inventories of predict usage patterns and inventories of raw materials.

### Example 5

### Rapid Prototyping Techniques

Most commercially available rapid prototyping machines use one of six techniques: stereolithography, laminated object manufacturing, selective laser sintering, fused deposition modeling, solid ground curing, and 3-D ink-jet printing. The different rapid prototyping techniques have their unique individual strengths. Because RP technologies are being increasingly used in non-prototyping, even production, applications, the techniques are often collectively referred to as solid free-form fabrication, computer automated manufacturing, or layered manufacturing. The latter term is particularly descriptive of the manufacturing process used by all commercial techniques. A software package “slices” the CAD model into a number of thin (˜0.1 mm) layers, which are then built up one atop another. Rapid prototyping is an “additive” process, conventionally combining layers of paper, wax, or plastic to create a solid object. In contrast, most machining processes (milling, drilling, grinding, etc.) are “subtractive” processes that remove material from a solid block. RP's additive nature allows it to create objects with complicated internal features that cannot be manufactured by other means. Below is a brief and general description of 6 rapid prototyping techniques. More description can be found at http://www.me.psu.edu/lamancusa/rapidpro/primer/chapter2.htm, and also described in U.S. Pat. Nos. 4,961,154, 5,198,159, 5,897,825, 6,508,971, and 6,790,403, the teachings of which are incorporated herein in their entirety to the extent not inconsistent with the teachings herein.

### Stereolithography

The technique builds three-dimensional objects (in this case masks or molds) from liquid photosensitive polymers that solidify when exposed to ultraviolet light. According to this technique, the model is conventionally built upon a platform situated just below the surface in a vat of liquid epoxy or acrylate resin. A low-power highly focused UV laser traces out the first layer, solidifying the object's cross section while leaving excess areas liquid. Next, an elevator incrementally lowers the platform into the liquid polymer. A sweeper re-coats the solidified layer with liquid, and the laser traces the second layer atop the first. This process is repeated until the prototype is complete. Afterwards, the solid part is removed from the vat and rinsed clean of excess liquid. Supports are broken off and the model is then placed in an ultraviolet oven for complete curing.

### Laminated Object Manufacturing

In this technique, layers of adhesive-coated sheet material are bonded together to form a prototype. The original material consists of paper laminated with heat-activated glue and rolled up on spools. A feeder/collector mechanism advances the sheet over the build platform, where a base has been constructed from paper and double-sided foam tape. Next, a heated roller applies pressure to bond the paper to the base. A focused laser cuts the outline of the first layer into the paper and then cross-hatches the excess area (the negative space in the prototype). Cross-hatching breaks up the extra material, making it easier to remove during post-processing. During the build, the excess material provides excellent support for overhangs and thin-walled sections. After the first layer is cut, the platform lowers out of the way and fresh material is advanced. The platform rises to slightly below the previous height, the roller bonds the second layer to the first, and the laser cuts the second layer. This process is repeated as needed to build the part, which will have a wood-like texture.

### Selective Laser Sintering

The technique uses a laser beam to selectively fuse powdered materials, such as nylon, elastomer, and metal, into a solid object. Parts are built upon a platform which sits just below the surface in a bin of the heat-fusable powder. A laser traces the pattern of the first layer, sintering it together. The platform is lowered by the height of the next layer and powder is reapplied. This process continues until the part is complete. Excess powder in each layer helps to support the part during the build. SLS machines are produced by DTM of Austin, Tex.

### Fused Deposition Modeling

In this technique, filaments of heated thermoplastic are extruded from a tip that moves in the x-y plane. Like a baker decorating a cake, the controlled extrusion head deposits very thin beads of material onto the build platform to form the first layer. The platform is maintained at a lower temperature, so that the thermoplastic quickly hardens. After the platform lowers, the extrusion head deposits a second layer upon the first. Supports are built along the way, fastened to the part either with a second, weaker material or with a perforated junction. Stratasys, of Eden Prairie, Minn. makes a variety of FDM machines ranging from fast concept modelers to slower, high-precision machines. Materials conventionally include ABS (standard and medical grade), elastomer (96 durometer), polycarbonate, polyphenolsulfone, and investment casting wax.

### Solid Ground Curing

Solid ground curing (SGC) is somewhat similar to stereolithography (SLA) in that both use ultraviolet light to selectively harden photosensitive polymers. Unlike SLA, SGC cures an entire layer at a time. First, photosensitive resin is sprayed on the build platform. Next, the machine develops a photomask (like a stencil) of the layer to be built. This photomask is printed on a glass plate above the build platform using an electrostatic process similar to that found in photocopiers. The photomask is then exposed to UV light, which only passes through the transparent portions of the photomask to selectively harden the shape of the current layer. After the layer is cured, the machine vacuums up the excess liquid resin and sprays wax in its place to support the model during the build. The top surface is milled flat, and then the process repeats to build the next layer. When the part is complete, it must be de-waxed by immersing it in a solvent bath. SGC machines are distributed in the U.S. by Cubital America Inc. of Troy, Mich.

### 3-D Ink-Jet Printing

Ink-Jet Printing refers to an entire class of machines that employ ink-jet technology. The first was 3D Printing (3DP), developed at MIT and licensed to Soligen Corporation, Extrude Hone, and others. The ZCorp 3D printer, produced by Z Corporation of Burlington, Mass. (www.zcorp.com) is an example of this technology. Parts are built upon a platform situated in a bin full of powder material. An ink jet printing head selectively deposits or “prints” a binder fluid to fuse the powder together in the desired areas. Unbound powder remains to support the part. The platform is lowered, more powder added and leveled, and the process repeated. When finished, the fused part is then removed from the unbound powder, and excess unbound powder is blown off. Finished parts can be infiltrated with wax, glue, or other sealants to improve durability and surface finish. Typical layer thicknesses are on the order of 0.1 mm.

3D Systems' (www.3dsystems.com) version of the ink jet based system is called the Thermo-Jet or Multi-Jet Printer. It uses a linear array of print heads to rapidly produce thermoplastic models. If the part is narrow enough, the print head can deposit an entire layer in one pass. Otherwise, the head makes several passes.

As alluded to above, in view of the teachings herein, the above rapid production techniques could be adapted to produce either masks or molds based on scanned images of a target region of a patient's face.

### Example 6

### Impression Tray and Method of Making Facial Impression

According to another embodiment, the invention pertains to a method of making a facial impression for purposes of using to fabricate a customized CPAP mask. The method comprises positioning of a tray over the nasal region of a subject. The nasal region comprises at least a portion of a subject's nose and nostrils. The tray is loaded with a deformable material. The tray is impressed upon the subject's face thereby leaving a facial impression in the deformable material in the tray. In a specific embodiment, the deformable material sets during and/or after the impression is formed so as to increase hardness and rigidity of the facial impression. This allows the deformable material to retain the geometry of the facial impression. The deformable material may be remove or left in the tray. The deformable material is sent to a central location for scanning and fabrication of a customized mask.

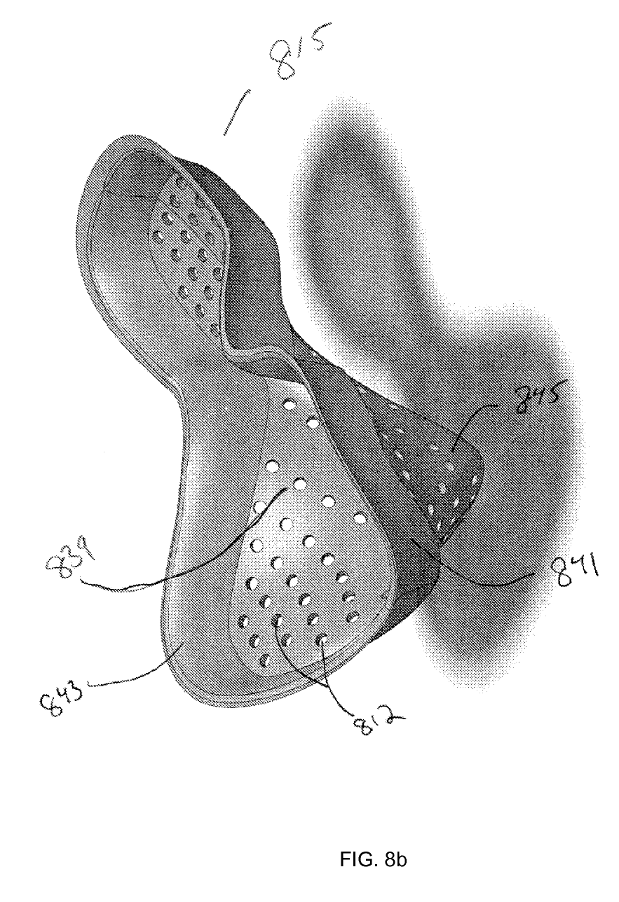

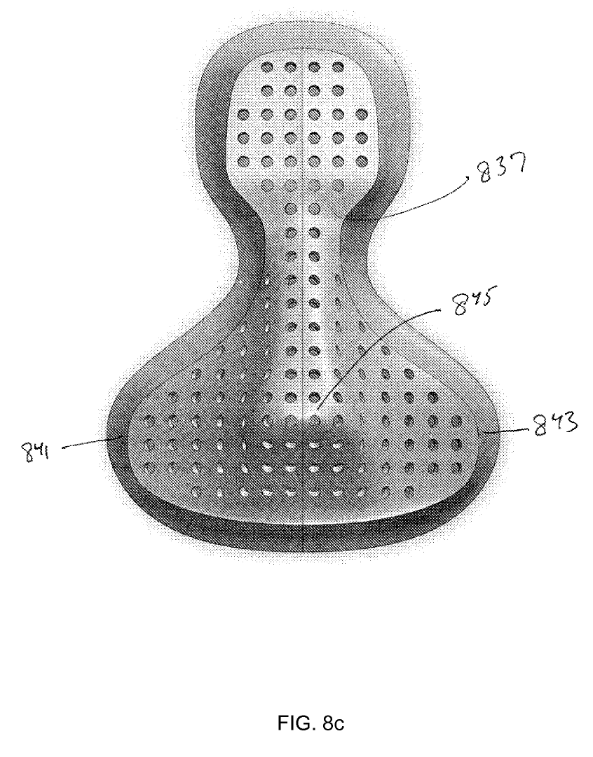

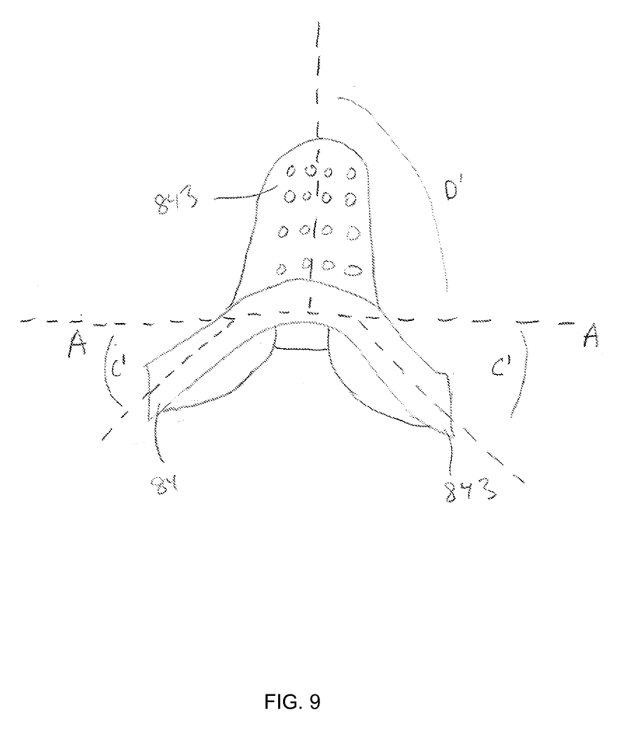

FIG. 8 a-c shows a specific embodiment 815 of a tray for obtaining a facial impression. The tray comprises a front side 837, a back side 838, a first lateral flap portion 841, a second lateral flap portion 843 and a protruding nose receptacle portion 845. Defined in the tray 815 are holes 812. The holes 812 allow for deformable material loaded in the tray 815 to escape when the tray 815 is impressed upon a subject's face. FIG. 9 shows a bottom planar view of the tray embodiment 815. The first and second flap portions 841, 843 possess a transverse angle C′ respective to a coronal cross-section A-A. The protruding nose receptacle portion 845 extends generally perpendicular D′ (80-100 degrees) respective to said coronal cross-section A-A.

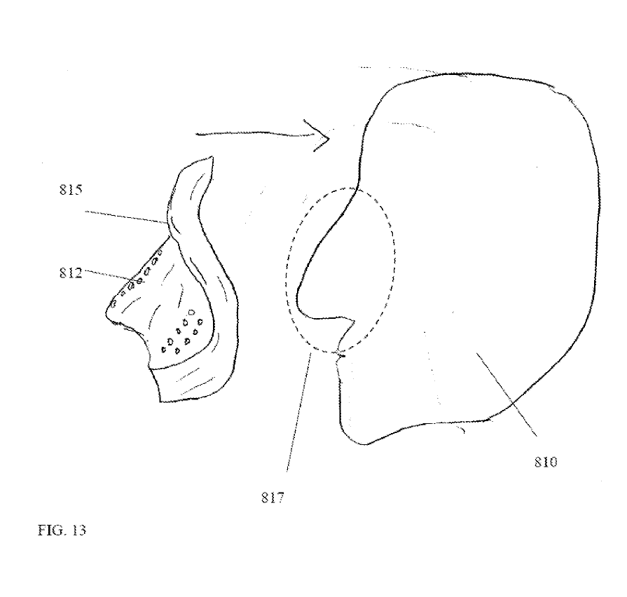

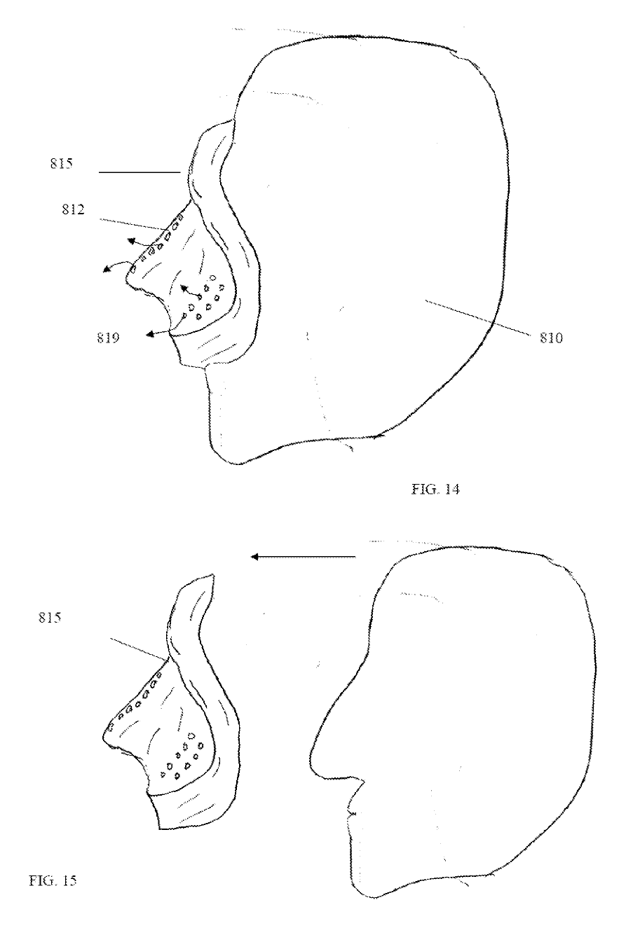

FIG. 13 shows a diagram of the tray 815 being applied to a subject 810. The tray 815 is configured to cover a nasal region 817 of the subject. The tray includes holes 812 and is loaded with a deformable material. FIG. 14 shows the tray 815 impressed upon the face of the subject 810. Deformable material 819 is displaced through holes 812 to accommodate the face of the subject 810. After the facial impression is formed in the deformable material in the tray 815, the tray 815 is removed from the subject 810, as shown in FIG. 15

Materials useful as deformable material include, but are not limited to, alginate, polyvinyl siloxanes, polyethers, rubber base, and zinc oxide eugenol pastes. High molecular weight poly (epsilon-caprolactone) (also known as “polycaprolactone”), various thermoplastic acrylics, and other heat-softenable materials have been used to take impressions of teeth or to make custom dental trays. References describing such thermoplastic compositions include U.S. Pat. Nos. 2,020,311, 4,227,877, 4,361,528, 4,401,616, 4,413,979, 4,569,342, 4,619,610 4,657,509, 4,659,786, 4,768,951, 4,776,792, and 4,835,203. Other references include Kokai (Japanese Published Pat. Appl.) Nos. Sho 63[1988]-96536 [which discloses oil-extended ethylene vinyl acetate (“EVA”) copolymers that are stored in syringes before use]; Sho 63[1988]-171554 and Sho 63[1988]-270759; and TONE®POLYMERS P-300 AND P-700 High Molecular Weight Caprolactone Polymers (1988 product literature of Union Carbide Corp.). In the main, the thermoplastic materials of these references are used without a surrounding shell, or their manner of use is not specifically described. In some instances the thermoplastic materials are themselves shaped to form a custom tray which is in turn used as a shell surrounding a conventional non-thermoplastic elastomeric impression material such as a polysiloxane. The thermoplastic materials of the '610 and '792 patents are exceptions; both employ a thin thermoplastic sheet surrounded by a rigid tray. In each of these latter two patents (and in the “OCCLUSAL HARMONY” dental tray from Advantage Dental Products, Inc., a commercial version of the tray shown in the '792 patent) the thin thermoplastic sheet is used to make a bite registration of only the occlusal tooth surfaces. Based on the teachings herein, those skilled in the art will appreciate how to adapt the materials described above for use in obtaining facial impressions.

Other commercially available dental impression trays that contain a heat-softenable thermoplastic include the “EASY TRAY” from Oral Dynamics Inc. Division of Anson International, the “HEAT FORM” tray from Shofu, Inc., and the “AQUERON” custom tray from Erkodent Company. These latter three products are heated and shaped to conform to the shape of a patient's dental tissue, then typically filled with elastomeric impression material and reinserted in the patient's mouth to take an impression of the dental tissue.

There are only a few preloaded impression trays (trays intended to be sold with the impression material in the tray) described in the dental literature. U.S. Pat. Nos. 4,553,936 and 4,867,680 describe preloaded trays containing light-curable acrylic impression materials. The impression materials are not said to be thermoplastic. Instead, they apparently are flowable or pourable at room temperature, and are held in the tray using a flexible cover sheet or skin material.

Kokai Hei 2[1990]-45049 describes an impression technique that employs a thermoplastic propionic cellulose acetate impression material that is heated in a rod shape, bent to conform to an arch-shaped tray, “piled” on the tray, and inserted into a patient's mouth while still warm. The tray is not a preloaded dental impression tray, in that the impression material resides in the tray for only a short time before it is inserted in a patient's mouth, and is not intended to be sold in preloaded form.

Other suitable materials as “deformable material” are those taught in U.S. Pat. Nos. 7,456,246; 3,070,566; 4,657,959; 4,877,854; 5,415,544; 5,580,921; 5,786,414; 5,849,812; 5,907,002; and 6,762,242.

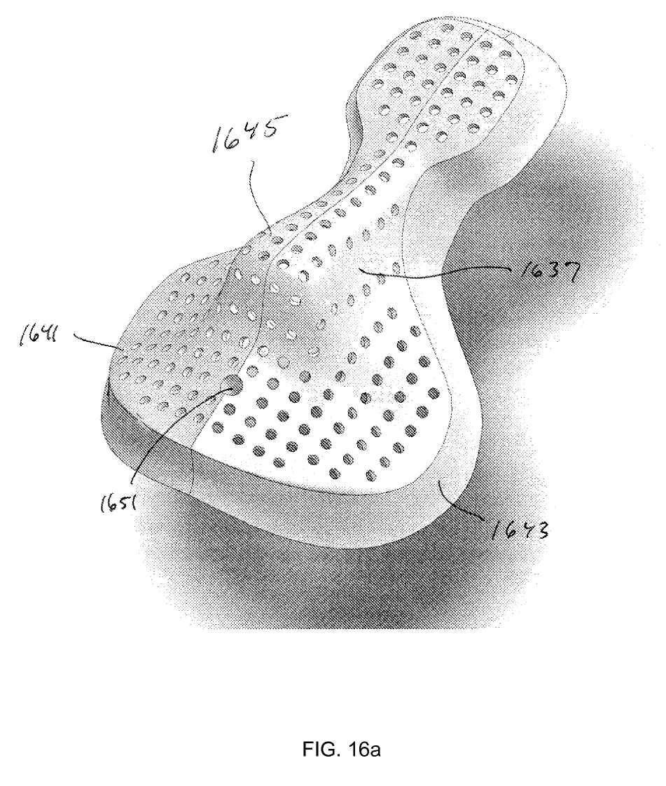

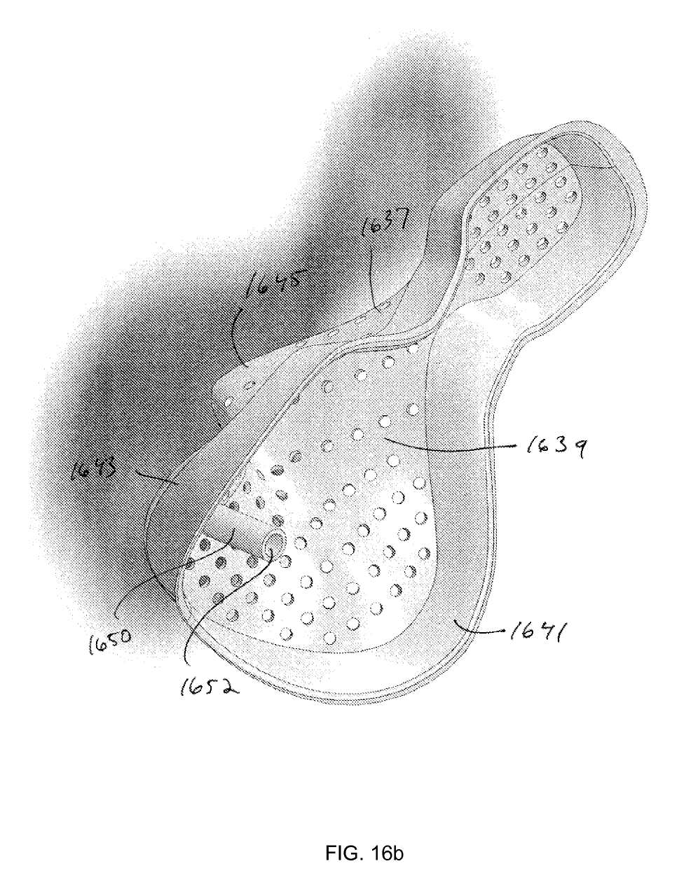

FIG. 16 shows an alternative tray embodiment 1615 (FIG. 16a front perspective, 16b back perspective) that is designed for obtaining a facial impression of a nasal and mouth region comprising at least a portion of a subject's nose, nostrils, and mouth. The tray 1615 comprises a front side 1637 and a back side 1639. The tray 1615 also comprises a first lateral flap 1641 and a second lateral flap 1643 and a protruding nose receptacle portion. Furthermore, the tray 1615 includes a conduit 1650 that communicates between the front side 1637 and back side 1639. The conduit 1650 having a first open end 1651 and a second open end 1652 thereby allowing the subject to breathe through it as the tray loaded with deformable material is impressed upon the subject's face.

### Example 7

### Deformable Blank (Mold) for Making Facial Impression

In another embodiment, the invention pertains to a unitary, deformable mold (also referred to herein as ‘blank’) that may be impressed upon a subject's face to form a facial impression. The deformable mold may be a unified block comprised of one or more layers of a foam or similar deformable material. Upon pressing the mold onto a subject's face, facial features such as nose and/or mouth and the nasal region push into the mold thereby forming an impression. The mold retains the impression upon removing the mold from the face. In a related embodiment, the mold may be a thin, planar mold (less than 1.5 inches thick) that wraps and conforms to the desired region of a subject's face. The molds enable the production of a facial impression which may then be sent to a remote location for making a model for scanning according the methods taught herein.

Molds of the feet may be used to form arch supports or orthopedic footwear (U.S. Pat. No. 4,006,542). Molds of a breast may be used to form a prosthesis after the breast is surgically removed (U.S. Pat. No. 4,086,666). Molds of other body parts have been described: fingers/fingernails (U.S. Pat. No. 4,361,160); scalp (U.S. Pat. No. 3,889,695); head/knee/groin/ear/breast (U.S. Pat. No. 4,006,542); feet/hands/face (U.S. Pat. No. 4,828,116). Perhaps the largest use of such molds is with amputated limbs (U.S. Pat. Nos. 4,225,982; 4,696,780; 4,923,474; 5,004,477). A mold of the “stump” of the amputated member is taken (forming a “negative” mold), and then used to cast a replica of the “stump” (a “positive” reproduction) which can then be used to fashion and fit a prosthesis for the amputated limb.

Prior methods have generally used plaster of Paris as the casting material to form such molds. This is usually applied as multiple strips of material impregnated with plaster of Paris, each of which must be soaked. This is time consuming and very messy. The plaster of Paris takes a long time to set (10 to 30 minutes or more), and complete drying may take up to 72 hours. The wet plaster of Paris must thus remain on the body part for a considerable period before it can be removed. The damp plaster is very weak and fragile when removed. Some patients may have difficulty remaining still for this extended period which may be uncomfortable for the patient. The prolonged setting time also causes loss of resolution in the mold—over a period of time, small body movements cause distortion or “blurring” of the mold surface, with loss of fine details. Plaster of Paris molds also tend to be heavy, to break easily and are subject to chipping.

Some applications have used alginate (U.S. Pat. Nos. 4,361,160 and 4,828,116) to form molds. The part to be molded must be immersed in a container of alginate and water. Thus the mold is large and heavy and not all body parts are amenable to immersion. This present issues of safety for immersing a person's face. The application is messy and requires various sizes of containers. Alginate is not available in substrate impregnated strips. Special mixers are often required for the alginate-water mixing, to eliminate air bubbles. The alginate molds are initially rubbery in texture. After prolonged periods they become very hard, and distorted due to water loss. Thus, they are not suitable for long term storage.

The mold may be made of materials known in the art that are used for taking impressions of other parts of the body, including, but not limited to those mentioned above for this Example and those discussed in Example 6. With respect to molds designed to take impressions of the foot and arch, the mold is formulated such that it is at least 5 percent softer than the molds presented used for feet and other limbs as described in the cited references.

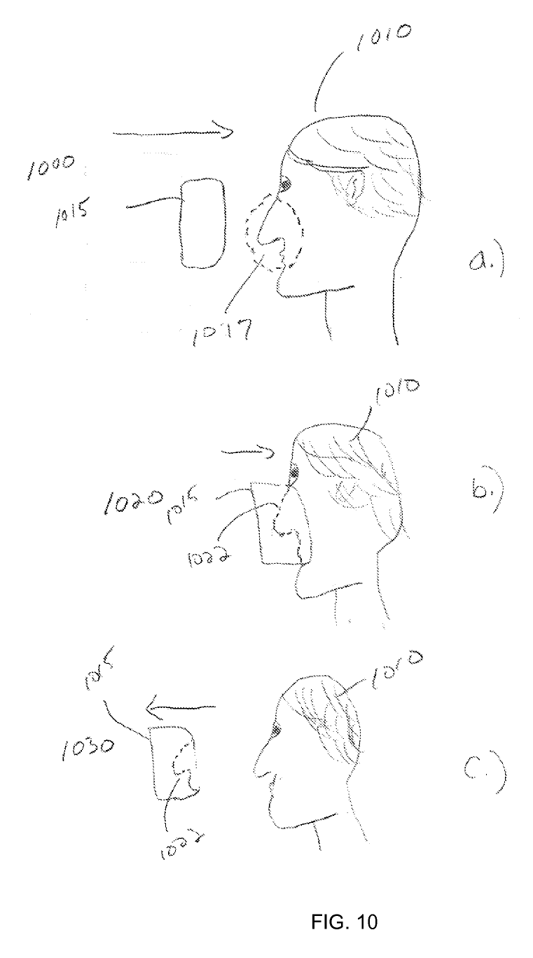

FIG. 10 shows an application of a mold embodiment 1015. FIG. 10a shows a first step 1000 before the mold 1015 is applied to the nasal region 1017 of the subject 1010. FIG. 10b shows a second step 1020 of the application of the mold 1015 to the subject 1010. The subject 1010 makes an impression 1022 in the mold showed by dashed lines. FIG. 10c shows the removal 1030 of the mold 1015 from the subject 1010. The impression 1020 remains in the mold 1015. The mold 1015 is then sent to a remote location for manufacturing of a model for scanning.

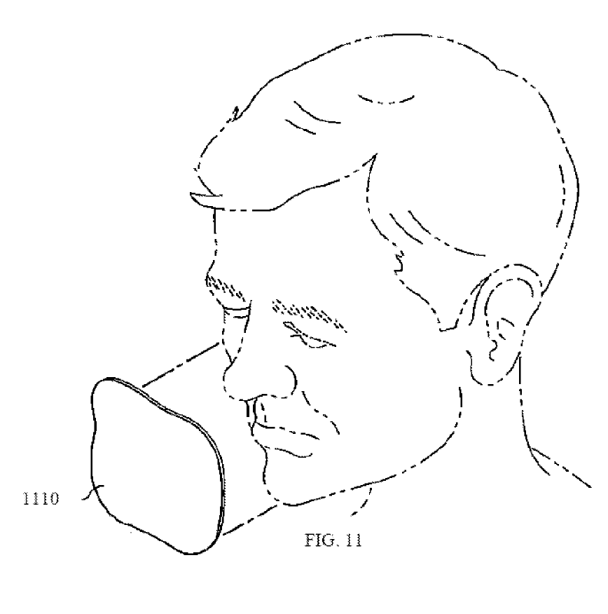

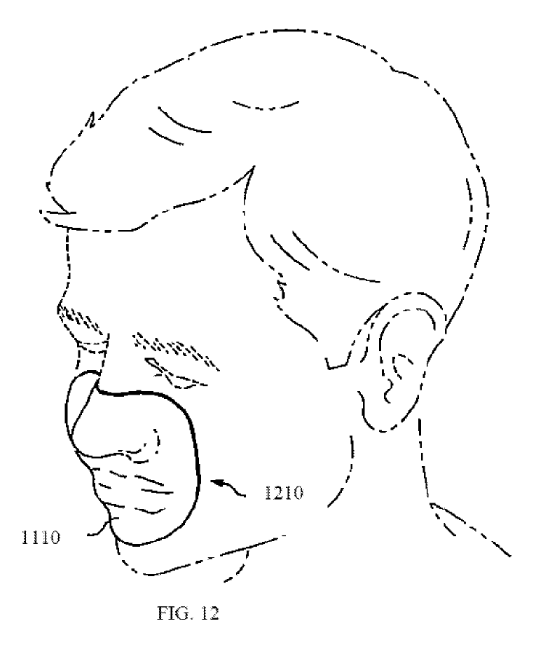

FIGS. 11 and 12 show an alternative embodiment 1110 that pertains to a thin, planar mold (less than 1.5 inches) designed for wrapping onto the face of a subject. FIG. 11 shows the mold 1110 before placement on the subject's face. FIG. 12 shows the mold 1110 on the subject's face 1210. The mold 1110 may be made of a moldable material having enough plasticity to impress upon the subject's face but enough structural integrity to retain the impression upon being removed from the subject. Alternatively, the mold 1110 is made of a curable material that is soft when applied and cures during or after removal from the subject.

### Example 8

### Use of White Light Scanning in Conjunction with High Definition Camera

According to another embodiment, the invention pertains to a system for rapidly designing a mask used in conjunction with CPAP that is anatomically customized to a particular region of a patient's face. The system includes a first camera and a second camera configured at different angles respective to said patient's face, each camera producing a first and second image, respectively. The system also includes a projector for projecting white light onto the patient's face. In a specific embodiment, the projector projects a pattern onto the face. In a more specific embodiment, the pattern is such that it allows triangulation processing by a computer. The system includes a first computer communicatingly connected to said projector and/or first and second cameras. Optionally, the system includes a second computer communicatingly connected to said first computer. The first computer includes a processing module and said optional second computer includes a processing module, and one or more computer program code modules stored on one or the other of said first and optional second computers, or both. The one or more computer program modules comprise a first computer program code module for processing the first image to form a first image dataset relating to the topography of the patient's face. Also included is a second computer program code module for processing the second image to form a second image dataset relating to the topography of the patient's face. Alternatively, the first program code module can process both the first and second images. The embodiment includes a third program code module for integrating the first and second image datasets to produce a multidimensional model image. Alternatively, the first and second image datasets are combined to form a master image dataset (such as a point cloud) that is then used to produce the multidimensional model image. Multidimensional model images include, but are not limited to, STL, NURBS, CAD or triangle mesh models. As discussed above, Furthermore, the embodiment may optionally include a fourth computer program code module for directing a CNC machine to produce a mold of said patient's face based on said multidimensional model image. In one embodiment, the computer used in this example is loaded with FlexScan3D software (3D3 Solutions, Vancouver, BC).

A related embodiment of the invention pertains to a method for rapidly producing a mask used in conjunction with CPAP that is anatomically customized to a particular region of a patient's face. The method involves projecting a pattern onto the patient's face. An image is obtained of the face with the pattern projected thereon by at least two cameras at separate locations. The images are processed based on the projected pattern, such as by a triangulation processing, to produce a multidimensional model image used in directing a cutting machine. In a further embodiment, a mold is cut by the cutting machine based on the multidimensional model image. A layer of material is then vacuformed over the mold to form an unfinished mask.

Structured-light 3D scanners project a pattern of light on the subject and look at the deformation of the pattern on the subject. The pattern may be one dimensional or two dimensional. An example of a one dimensional pattern is a line. The line is projected onto the subject using either an LCD projector or a halogen projector. A camera, offset slightly from the pattern projector, looks at the shape of the line and uses a technique similar to triangulation to calculate the distance of every point on the line. In the case of a single-line pattern, the line is swept across the field of view to gather distance information one strip at a time.

An example of a two-dimensional pattern is a grid or a line stripe pattern. A camera is used to look at the deformation of the pattern, and an algorithm is used to calculate the distance at each point in the pattern. Consider an array of parallel vertical laser stripes sweeping horizontally across a target. In the simplest case, one could analyze an image and assume that the left-to-right sequence of stripes reflects the sequence of the lasers in the array, so that the leftmost image stripe is the first laser, the next one is the second laser, and so on. In non-trivial targets having holes, occlusions, and rapid depth changes, however, this sequencing breaks down as stripes are often hidden and may even appear to change order, resulting in laser stripe ambiguity. This problem can be solved using algorithms for multistripe laser triangulation.

The advantage of structured-light 3D scanners is speed. Instead of scanning one point at a time, structured light scanners scan multiple points or the entire field of view at once. This reduces or eliminates the problem of distortion from motion. Some existing systems are capable of scanning moving objects in real-time.

Recently, a real-time scanner a using digital fringe projection and phase-shifting technique (a various structured light method) was developed, to capture, reconstruct, and render high-density details of dynamically deformable objects (such as facial expressions) at 40 frames per second.

There are several depth cues contained in the observed stripe patterns. The displacement of any single stripe can directly be converted into 3D coordinates. For this purpose, the individual stripe has to be identified, which can for example be accomplished by tracing or counting stripes (pattern recognition method). Another common method projects alternating stripe patterns, resulting in binary Gray code sequences identifying the number of each individual stripe hitting the object. An important depth cue also results from the varying stripe widths along the object surface. Stripe width is a function of the steepness of a surface part, i.e. the first derivative of the elevation. Stripe frequency and phase deliver similar cues and can be analyzed by a Fourier transform. Finally, the wavelet transform has recently been discussed for the same purpose.

In many practical implementations, series of measurements combining pattern recognition, Gray codes and Fourier transform are obtained for a complete and unambiguous reconstruction of shapes.

Another method also belonging to the area of fringe projection has been demonstrated, utilizing the depth of field of the camera ((Univ. of Stuttgart)). It is also possible to use projected patterns primarily as a means of structure insertion into scenes, for an essentially photogrammetric acquisition.

Point clouds are most often created by 3D scanners. These devices measure a large number of points on the surface of an object, and output a point cloud as a data file. The point cloud represents the visible surface of the object that has been scanned or digitized.

Point clouds themselves are generally not directly usable in most 3D applications, and therefore are usually converted to triangle mesh models, NURBS surface models (David F. Rogers: An Introduction to NURBS with Historical Perspective, Morgan Kaufmann Publishers 2001.), or CAD models. Techniques for converting a point cloud to a polygon mesh include Delaunay triangulation and more recent techniques such as Marching triangles, Marching cubes, and the Ball-Pivoting algorithm.

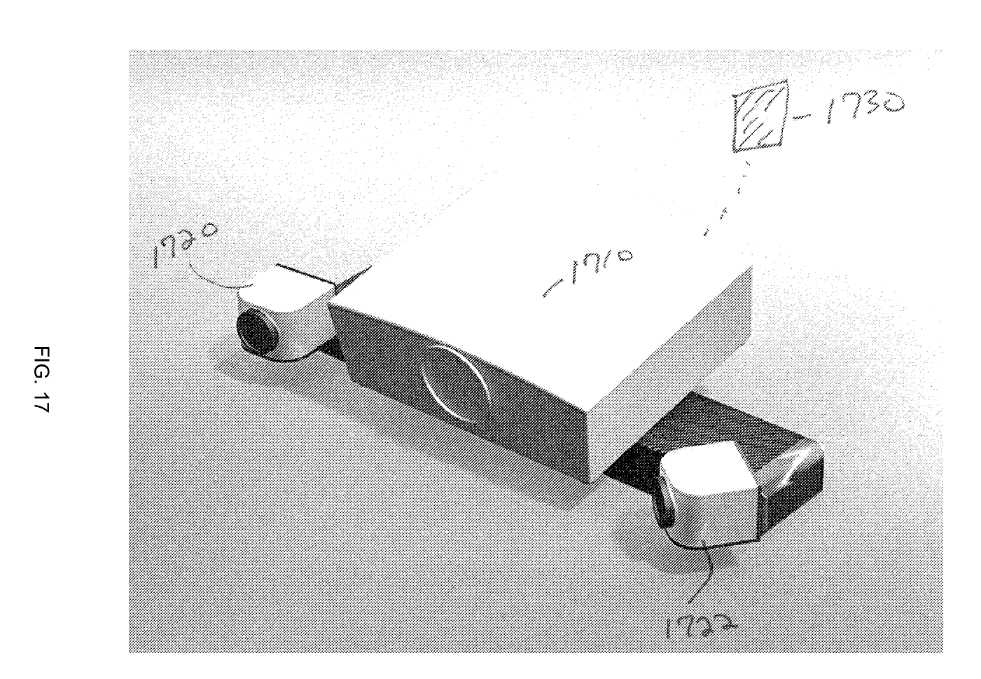

Turning to FIG. 17, what is shown is embodiment 1700 pertaining to a projector 1710 and a first camera 1720 and a second camera 1722. The projector 1710 is designed to project patterned white light onto a patient's face and the first and second cameras 1720, 1722 capture the images of the patterned light. In a specific embodiment, the patterned light comprises a series of stripes and the embodiment 1700 is enabled to use binary gray code sequences to identify each individual stripe hitting the patient's face. After all of the images are captured, the binary gray code is combined to create a depth map. From the depth map and calibration of the projector 1710 and cameras 1720,1722 a complete point cloud or mesh model of the patient's face, or portion thereof is created. Associated with the projector is a computer 1730 comprising at least one processing module and one or more program code modules for creating a point cloud and/or mesh model from images obtained and then creating a multidimensional model image from the point cloud and/or mesh model. The multidimensional model image is then modified to produce a protruding surface for producing a plenum during the cutting process as described above. The distance of the scanner system embodiment 1700 from a subject is optimized as well as the camera angles and relationship of the vertical and horizontal axes of the subject with those of the embodiment 1700.

Fechteler, P., Eisert, P., Rurainsky, J.: Fast and High Resolution 3D Face Scanning Proc. of ICIP 2007; and Peng, T., Gupta, S. K.: Model and algorithms for point cloud construction using digital projection patterns. Journal of Computing and Information Science in Engineering, 7(4): 372-381, 2007 are cited for background on producing structured light scanning and processing.

Those skilled in the art, in view of the teachings herein, will readily appreciate many other suitable materials which can be used and/or reformulated for purposes of making molds for obtaining a facial impression. U.S. Pat. Nos. 4,783,293; 4,006,542; 7,303,536; and 4,888,225 include a list of materials, in no way meant to be exhaustive, that may be implemented.

The inventor has realized that the proper fitting of certain mask embodiments can be improved by modifying the thicknesses and rigidity of certain portions of the mask. For example, the inventor has discovered that the mask will best be stabilized by adapting the rigidity of areas of the seal to correspond to the rigidity of facial area against which the mask rests. Therefore, certain embodiments of the mask comprise a higher rigidity on the seal where the mask rests upon the nasal bridge. Conversely, the mask seal is softer where it rests against the softer, fleshy portions of the patient's face around the sides and under the nose. Thus, another embodiment of the present invention relates to a mask have a seal that comprises a region have a different rigidity than another portion of the mask seal.

### Example 9

### Standard Mask with Customized Seal

In another embodiment, the invention is directed to a mask useful for administering CPAP, the mask including a standard mask body coupled with a customized mask seal. The customized mask seal closely simulates the geometry of the patient's face. The standard mask body is provided in several basic sizes and depending on the size and shape of the patient's face, the appropriate size is selected for conjoining to the customized mask seal. In one embodiment, a multidimensional scan of a patient's face is obtained, for example, by the procedures set forth in Examples 1-8 above. One or more scan images are used to produce a multidimensional model image of a portion of a patient's face. The multidimensional model image may then be used to produce a mold of a patient's face, or may be used to directly form the custom insert, such as directly through a CNC machine.

According to one embodiment, the invention relates to a method of making a customized mask useful for administering CPAP. The method includes obtaining one or more images of the patient's face and then generating a multidimensional model image of the patient's face by processing one or more images. The method then comprises contouring a mask seal blank to closely simulate the geometry of the patient's face to form a customized mask seal. Contouring may comprise cutting such as through use of a CNC machine or pressure molding (i.e. applying the mask seal blank to a mold of the patient's face). The customized mask seal is then attached to a standard mask body. In an alternative embodiment, the mask seal blank is associated with the standard mask body prior to the contouring step.

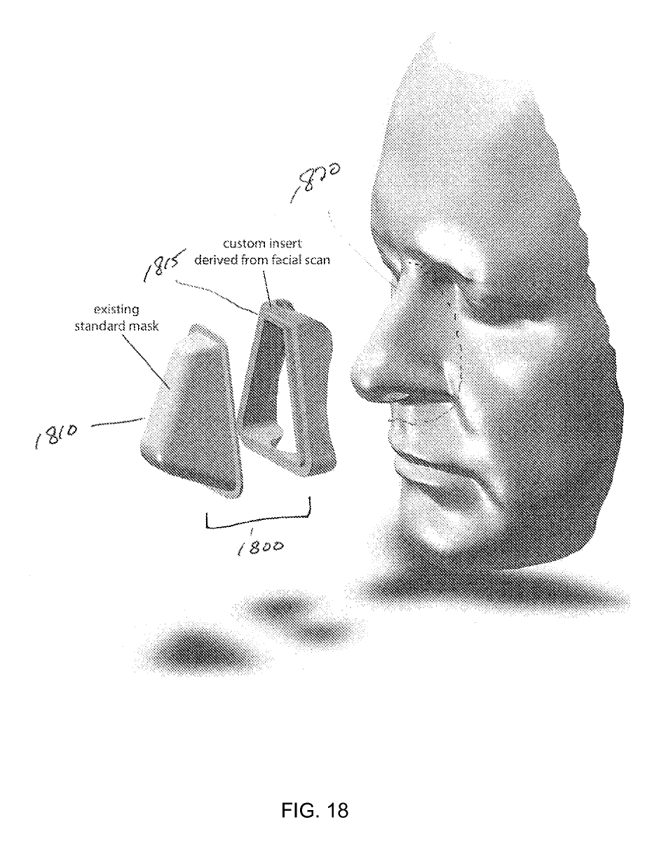

One version of an embodiment related to this Example is shown in FIG. 18. As shown a mask 1800 customized for a patient 1820 includes a standard mask body portion 1810 and a customized mask seal 1815. Standard mask bodies can be made en mass at a variety of different select sizes. Based on the anatomy of the patient 1820, the appropriate size can be selected for the customized mask seal 1815.

### Example 10

### Configured for Reduced Condensation Accumulation

The inventor has discovered a problem with conventional CPAP masks as well as a solution to this problem. Typically, CPAP is administered at night during a patient's sleeping hours. The air being inhaled and exhaled in the mask has a high humidity. The ambient temperature of most houses at night is between 70-79 degrees. Accordingly, the mask inner wall typically stabilizes at a temperature below the dew point of the air within the mask. This causes substantial condensation to develop on the inner wall of the mask, which results in the accumulation of undesired water within the mask over the course of the night. This presents an extremely uncomfortable situation for the patient and can run lead to the buildup of mildew and other microbes.

The inventor have developed a special insulating layer that is disposed either on the inner wall of the mask body, or alternatively, on the outer wall, or a layer on both inner and outer walls.

Thus, according to another embodiment, the invention pertains to a CPAP mask that includes a structural mask body component having an inner and outer wall, and at least one insulating layer disposed on either inner wall or outer wall, or both. The insulating layer may be comprised one of a number of materials including, but not limited to, isocyanurate, polyurethane, alkenyl aromatic polymers, polypropylenes, polyethylene terephthalates, polyethylenes and combinations thereof. In a more specific embodiment, the insulating layer is comprised of polyurethane.

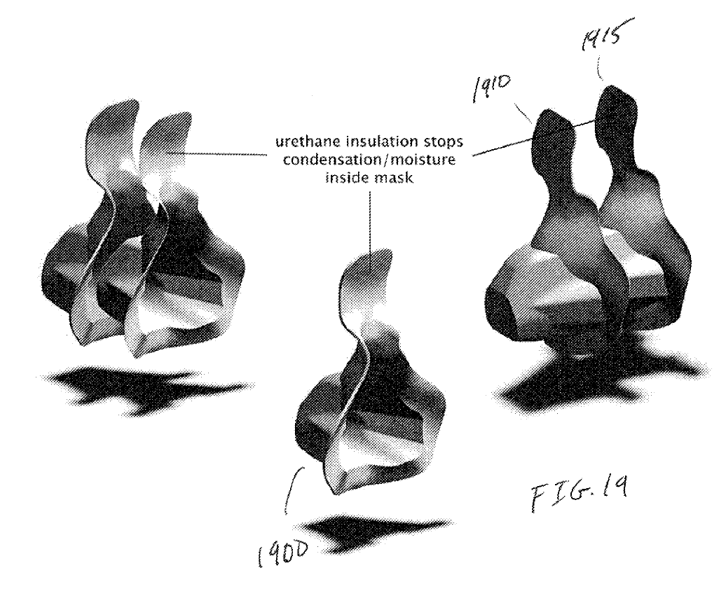

An additional embodiment of the invention relates to a mask useful for administering CPAP, the mask being produced using one or more scanned images of a patient's face and having a shape and dimension so as simulate a geometry of at least a portion of the patient's face. The customized mask comprises a mask body having an inner wall and an outer wall and a periphery. The customized mask further comprises a seal conjoined or integrated to the periphery of the mask body. FIG. 19 shows an example of a mask embodiment 1900 that includes a mask body portion 1910 and an insulating layer 1915.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference to the extent they are not inconsistent with the teachings herein.

1-20. (canceled)

21. A mask useful for administering CPAP to a patient, said mask comprising a standard mask body associated with a customized mask seal, said customized mask seal comprising a geometry that replicates contours of at least a portion of said patient's face.

22. The mask of claim 21, wherein said customized mask seal is made using a scan of said patient's face.

23. A method of making a mask useful for administering CPAP to a patient, the mask having a standard mask body portion and a customized mask seal, said method comprising
obtaining one or more images of at least a portion of said patient's face;
generating a multidimensional model of said at least one portion based on said one or more images;
creating a flexible mask seal that comprises a geometry replicating contours of said portion.

24. A mask useful for administering CPAP to a patient, said mask comprising a mask body having first layer comprised of a first material and a second layer comprised of a second material, wherein said second layer has a higher insulating potential than said first layer.

25. The mask of claim 24, wherein said second layer is comprised of isocyanurate, polyurethane, alkenyl aromatic polymers, polypropylenes, polyethylene terephthalates, or polyethylenes, or a combination thereof.
